(19) United States
(12) Patent Application Publication
(10) Pub. No.: US 2013/0253243 A1
(43) Pub. Date: Sep. 26, 2013

(54) METHODS FOR PRODUCING LINEAR PARAFFINS AND OLEFINS FROM NATURAL OILS

(71) Applicants: Andrea G. Bozzano, Northbrook, IL (US); Stanley Joseph Frey, Palatine, IL (US)

(72) Inventors: Andrea G. Bozzano, Northbrook, IL (US); Stanley Joseph Frey, Palatine, IL (US)

(73) Assignee: UOP LLC, Des Plaines, IL (US)

(21) Appl. No.: 13/427,706

(22) Filed: Mar. 22, 2012

(51) Int. Cl. C07C 1/20

(52) U.S. Cl. 585/324; 585/733

(57) ABSTRACT

A method for producing a linear paraffin includes providing a natural oil in a feed stream, deoxygenating the natural oil to form a stream comprising paraffins, purifying the stream comprising paraffins to form a purified stream comprising paraffins, and separating a first fraction of paraffin product from the purified stream comprising paraffins. A method for producing a linear olefin includes providing a natural oil in a feed stream, deoxygenating the natural oil to form a stream comprising paraffins, dehydrogenating the stream comprising paraffins to form a stream comprising olefins, purifying the stream comprising olefins to form a purified stream comprising olefins, and separating a first fraction of olefin product from the purified stream comprising olefins.

### FIELD OF THE INVENTION

The present invention relates generally to methods for producing renewable detergent compounds, and more particularly relates to methods for producing linear paraffins and olefins from natural oils.

### BACKGROUND OF THE INVENTION

While detergents made utilizing linear paraffin- and olefin-based surfactants are biodegradable, processes for creating linear paraffins and olefins are not based on renewable sources. Specifically, linear paraffins and olefins are currently produced from kerosene extracted from the earth. Due to the growing environmental concerns over fossil fuel extraction and economic concerns over exhausting fossil fuel deposits, there is a demand for using an alternate feed source for producing biodegradable surfactants for use in detergents and in other industries.

Accordingly, it is desirable to provide methods for producing linear paraffins and olefins from natural oils, i.e., oils that are not extracted from the earth. Furthermore, other desirable features and characteristics of the present invention will become apparent from the subsequent detailed description of the invention and the appended claims, when taken in conjunction with the accompanying drawing and this background of the invention.

### SUMMARY OF THE INVENTION

Methods for producing a linear paraffin or olefin product from a natural oil are provided herein. In accordance with an exemplary embodiment, a method for producing a linear paraffin includes providing a natural oil in a feed stream, deoxygenating the natural oil to form a stream comprising paraffins, purifying the stream comprising paraffins to form a purified stream comprising paraffins, and separating a first fraction of paraffin product from the purified stream comprising paraffins.

In another exemplary embodiment, a method for producing a linear olefin includes providing a natural oil in a feed stream, deoxygenating the natural oil to form a stream comprising paraffins, dehydrogenating the stream comprising paraffins to form a stream comprising olefins, purifying the stream comprising olefins to form a purified stream comprising olefins, and separating a first fraction of olefin product from the purified stream comprising olefins.

In accordance with yet another exemplary embodiment, a method for producing a linear paraffin and a linear olefin includes providing a natural oil in a feed stream, deoxygenating the natural oil to form a stream comprising paraffins, separating the stream comprising paraffins into a first portion comprising paraffins and a second portion comprising paraffins, purifying the first portion comprising paraffins to form a purified stream comprising paraffins, and separating a first fraction of paraffin product from the purified stream comprising paraffins. The method further includes dehydrogenating the second portion comprising paraffins to form a stream comprising olefins, purifying the stream comprising olefins to form a purified stream comprising olefins, and separating a first fraction of olefin product from the purified stream comprising olefins.

### BRIEF DESCRIPTION OF THE DRAWING

Embodiments of the present invention will hereinafter be described in conjunction with the following drawing FIGURE, wherein:

FIG. 1 schematically illustrates a system utilizing a process for producing linear paraffins and/or olefins from natural oils in accordance with an exemplary embodiment.

### DETAILED DESCRIPTION

The following Detailed Description is merely exemplary in nature and is not intended to limit the invention or the application and uses of the invention. Furthermore, there is no intention to be bound by any theory presented in the preceding Background or the following Detailed Description.

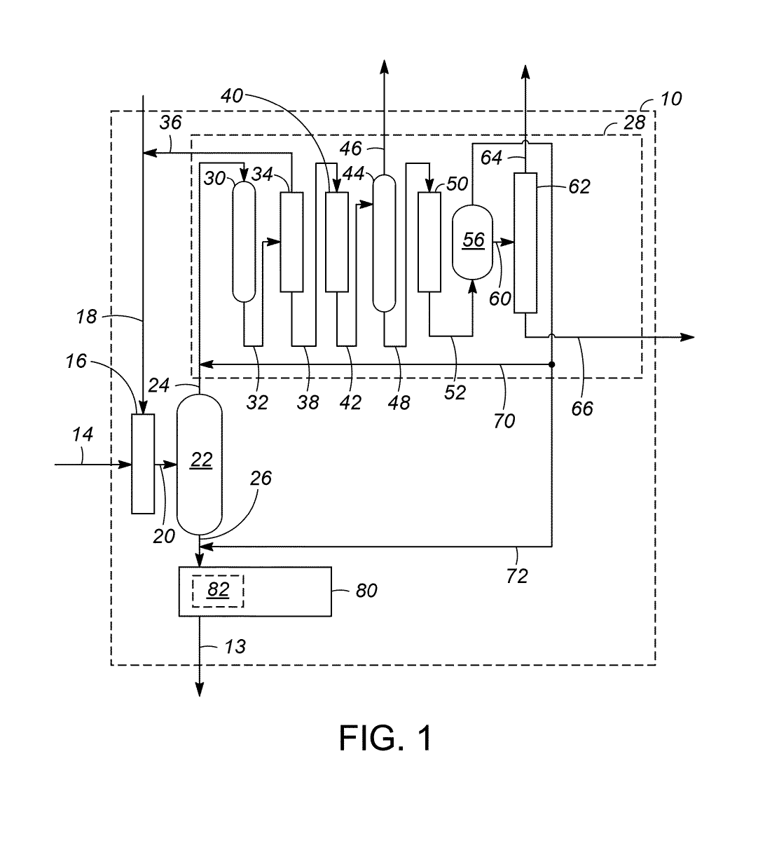

Various embodiments contemplated herein relate to methods and systems for producing a linear paraffin or olefin product from natural oils. In FIG. 1, an exemplary system 10 utilizing an exemplary process for producing a linear paraffin and/or olefin product from a natural oil feed 14. As used herein, natural oils are those derived from plant or algae matter, and are often referred to as renewable oils. Natural oils are not based on kerosene or other fossil fuels. In certain embodiments, the natural oils include, but are not limited to, one or more of coconut oil, babassu oil, castor oil, algae 1 byproduct, beef tallow oil, borage oil, camelina oil, Canola® oil, choice white grease, coffee oil, corn oil, Cuphea Viscosissima oil, evening primrose oil, fish oil, hemp oil, hepar oil, jatropha oil, Lesquerella Fendleri oil, linseed oil, Moringa Oleifera oil, mustard oil, neem oil, palm oil, perilla seed oil, poultry fat, rice bran oil, soybean oil, stillingia oil, sunflower oil, tung oil, yellow grease, cooking oil, and other vegetable, nut, or seed oils. Other natural oils will be known to those having ordinary skill in the art. The natural oils typically include triglycerides, free fatty acids, or a combination of triglycerides and free fatty acids, and other trace compounds.

In the illustrated embodiment, the natural oil feed 14 is delivered to a deoxygenation unit 16, which also receives a hydrogen feed 18. In the deoxygenation unit 16, the triglycerides and fatty acids in the feed 14 are deoxygenated and converted into linear paraffins. The deoxygenation unit 16 can be configured to catalytically deoxygenate the natural oils. Structurally, triglycerides are formed by three, typically different, fatty acid molecules that are bonded together with a glycerol bridge. The glycerol molecule includes three hydroxyl groups (HO—), and each fatty acid molecule has a carboxyl group (COOH). In triglycerides, the hydroxyl groups of the glycerol join the carboxyl groups of the fatty acids to form ester bonds. Therefore, during deoxygenation, the fatty acids are freed from the triglyceride structure and are converted into linear paraffins. The glycerol is converted into propane, and the oxygen in the hydroxyl and carboxyl groups is converted into either water or carbon dioxide. The deoxygenation reaction for fatty acids and triglycerides are illustrated, respectively, as:

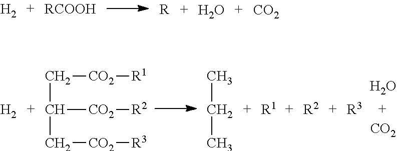

During the deoxygenation reaction, the length of a product paraffin chain Rⁿ will vary by a value of one depending on the exact reaction pathway. For example, if carbon dioxide is formed, then the chain will have one fewer carbon than the fatty acid source (Rⁿ). If water is formed, then the chain will match the length of the Rⁿ chain in the fatty acid source. Typically, due to the reaction kinetics, water and carbon dioxide are formed in roughly equal amounts, such that equal amounts of C_{X} paraffins and C_{X-1} paraffins are formed.

In FIG. 1, a deoxygenated stream 20 containing linear paraffins, water, carbon dioxide and propane exits the deoxygenation unit 16 and is fed to a separator 22. The separator 22 may be a multi-stage fractionation unit, distillation system, or similar known apparatus. In any event, the separator 22 removes the water, carbon dioxide, and propane from the deoxygenated stream 20. Further, the separator 22, or optionally another separator, may provide a means to separate the paraffins into various desirable fractions. For example, as shown in FIG. 1, a first portion of paraffins 24 and a second portion of paraffins 26 are illustrated, although any number of paraffin portions may be provided, depending on how many paraffin fractions are desired. In certain embodiments, the first portion of paraffins 24 has carbon chain lengths of C₁₀ to C₁₄. In other embodiments, the first portion of paraffins 24 has carbon chain lengths having a lower limit of C_{L}, where L is an integer from four (4) to thirty-one (31), and an upper limit of C_{U}, where U is an integer from five (5) to thirty-two (32). The second portion of paraffins 26 may have carbon chains shorter than, longer than, or a combination of shorter and longer than, the chains of the first portion of paraffins 24. In one particular embodiment, the first portion of paraffins 24 includes paraffins with C₁₀ to C₁₄ chains and the second portion of paraffins 26 includes paraffins with C₁₈ to C₂₀ chains.

Either or both paraffin portions 24 or 26 (or other portions if more are present) may thereafter be purified to remove trace contaminants, resulting in a purified paraffin product. In some embodiments, wherein only paraffin production is desired, the entire paraffin product (i.e., all of the one or more portions) may be purified at this stage. In other embodiments, some of the paraffin product is directed to further processing stages for the production of olefins. In still other embodiments, wherein only olefin production is desired, the entire paraffin product (i.e., all of the one or more portions) may be directed to further processing stages. As shown in the example embodiment illustrated in FIG. 1, the second paraffin portion 26 is directed to a purification system 80 to remove trace contaminants, such as oxygenates, nitrogen compounds, and sulfur compounds, among others. In one example, purification system 80 is an adsorption system. Alternatively or additionally, a PEP unit 82, available from UOP LLC, may be employed as part of purification system 80. Subsequent to purification, a purified paraffins stream 13 is removed from the system 10 as the paraffin product.

As further shown in FIG. 1, the first portion of paraffins 24 (i.e., that portion of linear paraffins directed for further processing to linear olefins, where desired) is introduced to a linear olefin production zone 28. Specifically, the first portion of paraffins 24 is fed into a dehydrogenation unit 30 in the olefin production zone 28. In the dehydrogenation unit 30, the first portion of paraffins 24 are dehydrogenated into mono-olefins of the same carbon numbers as the first portion of paraffins 24. Typically, dehydrogenation occurs through known catalytic processes, such as the commercially popular Pacol process. Conversion is typically less than 90%, leaving greater than 10% paraffins unconverted to olefins. Di-olefins (i.e., dienes) and aromatics are also produced as an undesired result of the dehydrogenation reactions as expressed in the following equations:

Mono-olefin formation: C_{X}H_{2X+2}→C_{X}H_{2X}+H₂

Di-olefin formation: C_{X}H_{2X}→C_{X}H_{2X-2}+H₂

Aromatic formation: C_{X}H_{2X-2}→C_{X}H_{2X-6}+2H₂

In FIG. 1, a dehydrogenated stream 32 exits the dehydrogenation unit 30 comprising mono-olefins and hydrogen, unconverted paraffins, as well as some byproduct di-olefins and aromatics. The dehydrogenated stream 32 is delivered to a phase separator 34 for removing the hydrogen from the dehydrogenated stream 32. As shown, the hydrogen exits the phase separator 34 in a recycle stream of hydrogen 36 that can, in some embodiments, be added to the hydrogen feed 18 to support the deoxygenation process upstream.

At the phase separator 34, a liquid stream 38 is formed and includes the mono-olefins, the unconverted paraffins, and any di-olefins and aromatics formed during dehydrogenation. The liquid stream 38 exits the phase separator 34 and enters a selective hydrogenation unit 40. In one exemplary embodiment, the hydrogenation unit 40 is a DeFine® reactor (or a reactor employing a DeFine® process), available from UOP LLC. The hydrogenation unit 40 selectively hydrogenates at least a portion of the di-olefins in the liquid stream 38 to form additional mono-olefins. As a result, an enhanced stream 42 is formed with an increased mono-olefin concentration.

As shown, the enhanced stream 42 passes from the hydrogenation unit 40 to a lights separator 44, such as a stripper column, which removes a light end stream 46 containing any light hydrocarbons, such as butane, propane, ethane and methane, that resulted from cracking or other reactions during upstream processing. With the light hydrocarbons 46 removed, stream 48 is formed and may be delivered to an aromatic removal apparatus 50, such as a PEP unit available from UOP LLC. As indicated by its name, the aromatic removal apparatus 50 removes aromatics from the stream 48 and forms a stream of mono-olefins and unconverted paraffins 52.

In a further processing step, the unconverted paraffins are separated from the olefins using a separator 56. In one particular embodiment, the separator 56 is an Olex® separator, available from UOP LLC. The Olex® process involves the selective adsorption of a desired component (i.e., olefins) from a liquid-phase mixture by continuous contacting with a fixed bed of adsorbent. In another particular embodiment, the separator 56 is a direct sulfonation separator. The separated, unconverted paraffins may optionally be directed back to the second paraffin portion 26 for purification (stream 72) and/or back to the first paraffin portion 24 for dehydrogenation for conversion to olefins (stream 70).

In FIG. 1, an olefins stream 60 exits the separator 56 and is fed to a separator 62. The separator 62 may be a multi-stage fractionation unit, distillation system, or similar known apparatus. The separator 62 may provide a means to separate the olefins into various desirable fractions. For example, as shown in FIG. 1, a first portion of olefins 64 and a second portion of olefins 66 are illustrated, although any number of olefin portions may be provided, depending on how many olefin fractions are desired. In certain embodiments, the first portion of olefins 64 has carbon chain lengths of C₁₀ to C₁₄. In other embodiments, the first portion of olefins 64 has carbon chain lengths having a lower limit of C_{L}, where L is an integer from four (4) to thirty-one (31), and an upper limit of C_{U}, where U is an integer from five (5) to thirty-two (32). The second portion of olefins 66 may have carbon chains shorter than, longer than, or a combination of shorter and longer than, the chains of the first portion of olefins 64. In one particular embodiment, the first portion of olefins 64 includes olefins with C₁₀ to C₁₄ chains and the second portion of olefins 66 includes olefins with C₁₈ to C₂₀ chains. Subsequent to separation, the purified olefins portions 64 and 66 are removed from the system 10 as the olefin product.

With reference now to exemplary natural oil feeds 14, in certain embodiments, the feed 14 is substantially homogeneous and includes free fatty acids within a desired range. For instance, the feed may be palm fatty acid distillate (PFAD). Alternatively, the feed 14 may include triglycerides and free fatty acids that all have carbon chain lengths appropriate for a desired alkylbenzene product 12.

In certain embodiments, the natural oil source is castor, and the feed 14 includes castor oils. Castor oils consist essentially of C₁₈ fatty acids with additional, internal hydroxyl groups at the carbon-12 position. For instance, the structure of a castor oil triglyceride is:

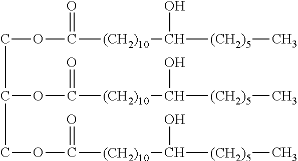

During deoxygenation of a feed 14 comprising castor oil, it has been found that some portion of the carbon chains are cleaved at the carbon-12 position. Thus, deoxygenation creates a group of lighter paraffins having C₁₀ to C₁₁ chains resulting from cleavage during deoxygenation, and a group of non-cleaved heavier paraffins having C₁₇ to C₁₈ chains. The lighter paraffins may form the first portion of paraffins 24 and the heavier paraffins may form the second portion of paraffins 26. It should be noted that while castor oil is shown as an example of an oil with an additional internal hydroxyl group, others may exist. Also, it may be desirable to engineer genetically modified organisms to produce such oils by design. As such, any oil with an internal hydroxyl group may be a desirable feed oil.

While at least one exemplary embodiment has been presented in the foregoing Detailed Description, it should be appreciated that a vast number of variations exist. It should also be appreciated that the exemplary embodiment or exemplary embodiments are only examples, and are not intended to limit the scope, applicability, or configuration of the invention in any way. Rather, the foregoing Detailed Description will provide those skilled in the art with a convenient road map for implementing an exemplary embodiment of the invention, it being understood that various changes may be made in the function and arrangement of elements described in an exemplary embodiment without departing from the scope of the invention as set forth in the appended Claims and their legal equivalents.

What is claimed is:

1. A method for producing a linear paraffin product from a natural oil comprising:
providing a natural oil in a feed stream;
deoxygenating the natural oil to form a stream comprising paraffins;
purifying the stream comprising paraffins to form a purified stream comprising paraffins; and
separating a first fraction of paraffin product from the purified stream comprising paraffins.

2. The method of claim 1, wherein deoxygenating the natural oil comprises catalytically deoxygenating the natural oil.

3. The method of claim 1, further comprising removing one or more of water, carbon dioxide, and propane from the stream comprising paraffins.

4. The method of claim 1, wherein separating the first fraction of paraffin product comprises separating a C₁₀-C₁₄ fraction of paraffin product.

5. The method of claim 1, further comprising separating a second fraction of paraffin product from the purified stream comprising paraffins.

6. The method of claim 5, wherein separating the second fraction of paraffin product comprises separating a C₁₈-C₂₀ fraction of paraffin product.

7. The method of claim 1, wherein purifying the stream comprising paraffins comprises purifying the stream comprising paraffins using an adsorption process.

8. The method of claim 1, wherein purifying the stream comprising paraffins comprises removing one or more of oxygenates, nitrogen compounds, and sulfur.

9. The method of claim 1, wherein providing a natural oil in a feed stream comprises providing a natural oil chosen from the group comprising: coconut oil, babassu oil, castor oil, algae 1 byproduct, beef tallow oil, borage oil, camelina oil, Canola® oil, choice white grease, coffee oil, corn oil, Cuphea Viscosissima oil, evening primrose oil, fish oil, hemp oil, hepar oil, jatropha oil, Lesquerella Fendleri oil, linseed oil, Moringa Oleifera oil, mustard oil, neem oil, palm oil, perilla seed oil, poultry fat, rice bran oil, soybean oil, stillingia oil, sunflower oil, tung oil, yellow grease, cooking oil, and mixtures thereof.

10. A method for producing a linear olefin product from a natural oil comprising:
providing a natural oil in a feed stream;
deoxygenating the natural oil to form a stream comprising paraffins;
dehydrogenating the stream comprising paraffins to form a stream comprising olefins; and
purifying the stream comprising olefins to form a purified stream comprising olefins.

11. The method of claim 10, further comprising separating a first fraction of olefin product from the purified stream comprising olefins.

12. The method of claim 1, further comprising separating a second fraction of olefin product from the purified stream comprising olefins.

13. The method of claim 12, wherein separating the first fraction of olefin product comprises separating a C₁₀-C₁₄ fraction of olefin product and wherein separating the second fraction of olefin product comprises separating a C₁₈-C₂₀ fraction of olefin product.

14. The method of claim 10, further comprising separating hydrogen from the stream comprising olefins and recycling the hydrogen for use in deoxygenating the natural oil.

15. The method of claim 10, further comprising separating olefins from the purified stream comprising olefins, wherein separating olefins from the purified stream comprising olefins comprises separating olefins using direct sulfonation.

16. The method of claim 10, further comprising separating olefins from the purified stream comprising olefins, wherein separating olefins from the purified stream comprising olefins comprises separating olefins using selective adsorption from a liquid phase mixture by continuous contact with a fixed-bed adsorbent.

17. The method of claim 16, further comprising recycling the unconverted paraffins for dehydrogenation to form olefins.

18. The method of claim 10, further comprising hydrogenating di-olefins in the stream comprising olefins using a selective hydrogenation process.

19. A method for producing a linear paraffin product and a linear olefin product from a natural oil comprising:
providing a natural oil in a feed stream;
deoxygenating the natural oil to form a stream comprising paraffins;
separating the stream comprising paraffins into a first portion comprising paraffins and a second portion comprising paraffins
purifying the first portion comprising paraffins to form a purified stream comprising paraffins;
separating a first fraction of paraffin product from the purified stream comprising paraffins;
dehydrogenating the second portion comprising paraffins to form a stream comprising olefins; and
purifying the stream comprising olefins to form a purified stream comprising olefins.

20. The method of claim 19, further comprising separating a first fraction of olefin product from the purified stream comprising olefins.
